(19) United States
(12) Patent Application Publication
(10) Pub. No.: US 2018/0028254 A1
(43) Pub. Date: Feb. 1, 2018

(54) THERAPEUTIC ENERGY APPLYING STRUCTURE AND MEDICAL TREATMENT DEVICE

(71) Applicant: OLYMPUS CORPORATION, Tokyo (JP)

(72) Inventor: Hiroki KAZUNO, Tokyo (JP)

(73) Assignee: OLYMPUS CORPORATION, Tokyo (JP)

(21) Appl. No.: 15/725,337

(22) Filed: Oct. 5, 2017

Related U.S. Application Data
(63) Continuation of application No. PCT/JP2015/065311, filed on May 27, 2015.

(51) Int. Cl. A61B 18/08

(52) U.S. Cl. CPC A61B 18/082; A61B 2018/00083

(57) ABSTRACT

A therapeutic energy applying structure includes: an insulating substrate; an electric resistance pattern provided on one surface of the substrate to generate heat; a connection portion provided on the one surface and configured to be electrically connected to the resistance pattern, the connection portion having a lower electric resistance than the resistance pattern; and a heat transfer plate facing the one surface and configured to transfer the heat from the resistance pattern to body tissue. The substrate and the heat transfer plate have an elongated shape extending in a same direction. The resistance pattern and the connection portion are arranged side by side in a longitudinal direction of the substrate. The substrate has a first region on which the connection portion is provided and has a second region on which the resistance pattern is provided. A width of the first region is larger than a width of the second region.

### CROSS-REFERENCE TO RELATED APPLICATION

This application is a continuation of International Application No. PCT/JP2015/065311, filed on May 27, 2015, the entire contents of which are incorporated herein by reference.

### BACKGROUND

### 1. Technical Field

The disclosure relates to a therapeutic energy applying structure and a medical treatment device.

### 2. Related Art

Conventionally, medical treatment devices having a therapeutic energy applying structure to apply energy to body tissue for treatment (such as connection (or anastomosis) and dissection) have been known (see JP 2014-124491 A).

The therapeutic energy applying structure described in JP 2014-124491 A includes a flexible substrate and a heat transfer plate to be described below.

The flexible substrate functions as a sheet heater. On one surface of the flexible substrate, an electric resistance pattern for generating heat by applying current and a connection portion connected to the electric resistance pattern by conduction are formed.

The heat transfer plate is configured using a conductor such as copper. Further, the heat transfer plate is disposed to face one surface (the electric resistance pattern) of the flexible substrate, and transfers the heat from the resistance pattern to the body tissue (applies heat energy to the body tissue).

Here, the flexible substrate is longer than the heat transfer plate, and one end side (the side on which the connection portion is provided) thereof protrudes from the heat transfer plate when being assembled. Further, a lead wire to supply power to the electric resistance pattern is connected to the connection portion provided on the one end side. That is, reduction in thickness is acquired by positioning the lead wire on one surface (the side on which the heat transfer plate is disposed) of the flexible substrate in the therapeutic energy applying structure described in JP 2014-124491 A.

### SUMMARY

In some embodiments, a therapeutic energy applying structure includes: an insulating substrate; an electric resistance pattern provided on one surface of the insulating substrate and configured to generate heat by applying current; a connection portion provided on the one surface of the insulating substrate and configured to be electrically connected to the electric resistance pattern, the connection portion having a lower electric resistance value than the electric resistance pattern; and a heat transfer plate disposed so as to face the one surface of the insulating substrate and configured to transfer the heat from the electric resistance pattern to a body tissue. Each of the insulating substrate and the heat transfer plate has an elongated shape extending in a same direction. The electric resistance pattern and the connection portion are arranged side by side in a longitudinal direction of the insulating substrate. The insulating substrate has a first region on which the connection portion is provided and has a second region on which the electric resistance pattern is provided. A width of the first region is larger than a width of the second region. The heat transfer plate covers an entire region of the electric resistance pattern when viewed from a thickness direction of the heat transfer plate. One end of the heat transfer plate in the longitudinal direction matches a boundary position between the electric resistance pattern and the connection portion, or the one end of the heat transfer plate is located on the connection portion deviating from the boundary position.

In some embodiments, a medical treatment device includes the therapeutic energy applying structure.

In some embodiments, a therapeutic energy applying structure includes: an insulating substrate; an electric resistance pattern provided on one surface of the insulating substrate and configured to generate heat by applying current; a connection portion provided on the one surface of the insulating substrate and configured to be electrically connected to the electric resistance pattern, the connection portion having a lower electric resistance value than the electric resistance pattern; and a heat transfer plate disposed so as to face the one surface of the insulating substrate and configured to transfer the heat from the electric resistance pattern to a body tissue. Each of the insulating substrate and the heat transfer plate has an elongated shape extending in a same direction. The electric resistance pattern and the connection portion are arranged side by side in a longitudinal direction of the insulating substrate. The insulating substrate has a first region on which the connection portion is provided and has a second region on which the electric resistance pattern is provided. A width of the first region is larger than a width of the second region.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic view illustrating a medical treatment system according to a first embodiment of the present invention;

FIG. 2 is an enlarged view of a distal end portion of the medical treatment device illustrated in FIG. 1;

FIG. 3 is a schematic view illustrating the therapeutic energy applying structure illustrated in FIG. 2;

FIG. 4 is a schematic view illustrating the therapeutic energy applying structure illustrated in FIG. 2;

FIG. 5 is a schematic view illustrating a positional relationship between a heat transfer plate, a flexible substrate, and an adhesive sheet illustrated in FIG. 3 or 4;

FIG. 6 is a schematic view illustrating a part of a therapeutic energy applying structure according to a second embodiment of the present invention;

FIG. 7 is a schematic view illustrating a part of a therapeutic energy applying structure according to a third embodiment of the present invention;

FIG. 8 is a schematic view illustrating a part of a therapeutic energy applying structure according to a fourth embodiment of the present invention;

FIG. 9 is a schematic view illustrating a part of a therapeutic energy applying structure according to a fifth embodiment of the present invention;

FIG. 10 is a side view illustrating a flexible substrate forming a therapeutic energy applying structure according to a sixth embodiment of the present invention;

FIG. 11 is a side view illustrating a flexible substrate forming a therapeutic energy applying structure according to a seventh embodiment of the present invention;

FIG. 12 is a side view illustrating a flexible substrate forming a therapeutic energy applying structure according to an eighth embodiment of the present invention;

FIG. 13 is a side view illustrating a therapeutic energy applying structure according to a ninth embodiment of the present invention; and

FIG. 14 is a schematic view illustrating a modified example of the ninth embodiment of the present invention.

### DETAILED DESCRIPTION

Exemplary embodiments of the present invention will be described below with reference to the drawings. The present invention is not limited to the embodiments to be described below. The same reference signs are used to designate the same elements throughout the drawings.

### First Embodiment

Schematic Configuration of Medical Treatment System

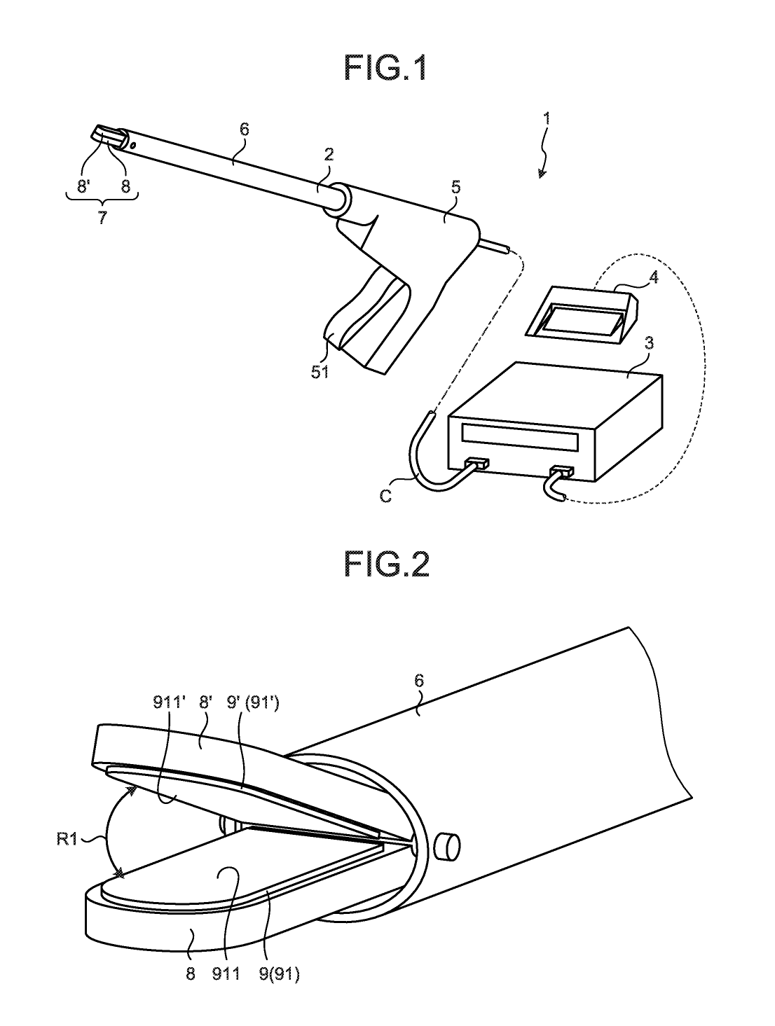

FIG. 1 is a schematic view illustrating a medical treatment system 1 according to a first embodiment of the present invention.

The medical treatment system 1 is configured to apply energy to a body tissue as a treatment target to perform treatment (such as connection (or anastomosis) and dissection) on the body tissue. As illustrated in FIG. 1, the medical treatment system 1 includes a medical treatment device 2, a control device 3, and a foot switch 4.

Configuration of Medical Treatment Device

The medical treatment device 2 is, for example, a linear type surgical medical treatment tool for performing treatment on a body tissue through an abdominal wall. As illustrated in FIG. 1, the medical treatment device 2 includes a handle 5, a shaft 6, and a grasping portion 7.

The handle 5 is a part to be gripped by an operator. As illustrated in FIG. 1, the handle 5 is provided with an operation knob 51.

As illustrated in FIG. 1, the shaft 6 has a substantially cylindrical shape, and one end thereof is connected to the handle 5. The grasping portion 7 is attached to the other end of the shaft 6. Further, an opening and closing mechanism (not illustrated), which opens and closes holding members 8 and 8′ (FIG. 1) forming the grasping portion 7 according to an operation of the operation knob 51 performed by the operator, is provided inside the shaft 6. An electric cable C (FIG. 1) connected to the control device 3 is disposed from one end side to the other end side via the handle 5 inside the shaft 6.

Configuration of Grasping Portion

FIG. 2 is an enlarged view of a distal end portion of the medical treatment device 2.

A pair of elements, which is denoted by the same reference numerals and distinguished by a prime mark (′) throughout the drawings, shares the same configuration.

The grasping portion 7 is a part for grasping a body tissue to treat the body tissue. As illustrated in FIG. 1 or 2, the grasping portion 7 includes the pair of holding members 8 and 8′.

The pair of holding members 8 and 8′ is pivotally supported at the other end of the shaft 6 so as to be capable of being opened and closed in a direction of an arrow R1 (FIG. 2), and enables grasping of the body tissue according to the operation of the operation knob 51 performed by the operator.

Therapeutic energy applying structures 9 and 9′ are provided on the pair of holding members 8 and 8′, respectively, as illustrated in FIG. 2.

Since the therapeutic energy applying structures 9 and 9′ have the same configuration, only the therapeutic energy applying structure 9 will be described hereinafter.

Configuration of Therapeutic Energy Applying Structure

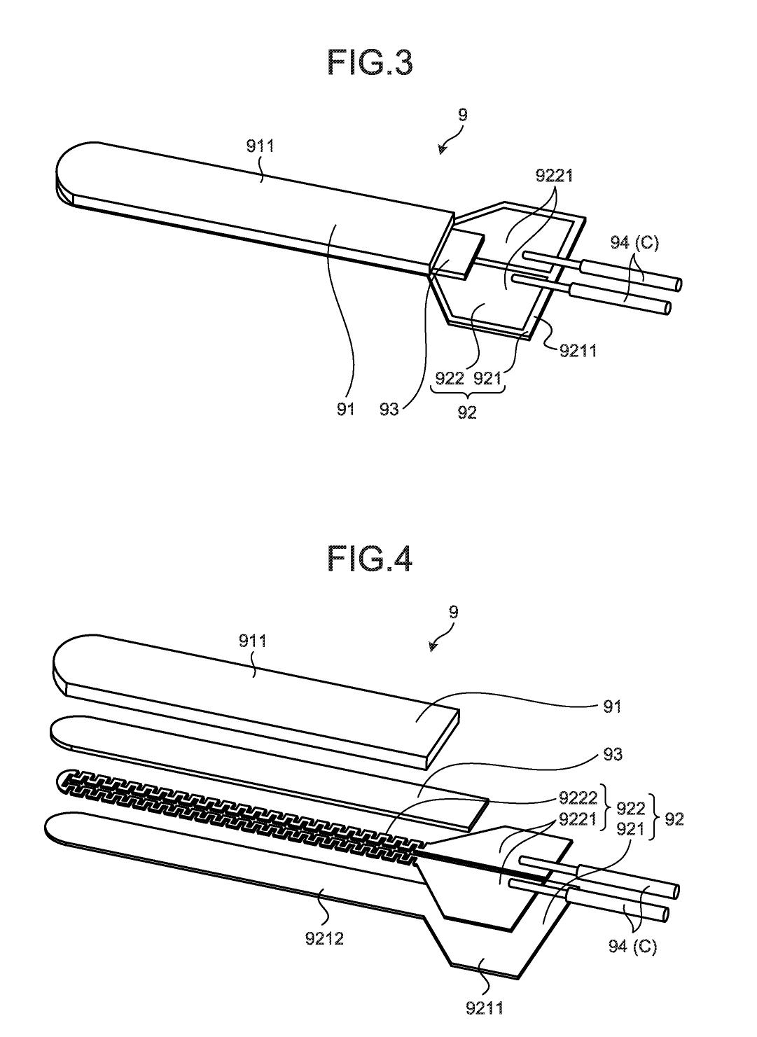

FIGS. 3 and 4 are schematic views illustrating the therapeutic energy applying structure 9. Specifically, FIG. 3 is a perspective view of the therapeutic energy applying structure 9 viewed from an upper side in FIG. 2. FIG. 4 is an exploded perspective view of FIG. 3.

The therapeutic energy applying structure 9 is attached to a surface of the holding member 8 on the upper side disposed on a lower side in FIGS. 1 and 2. The therapeutic energy applying structure 9 applies heat energy to the body tissue under control of the control device 3. As illustrated in FIG. 3 or 4, the therapeutic energy applying structure 9 includes a heat transfer plate 91, a flexible substrate 92, an adhesive sheet (adhesive layer) 93, and two lead wires 94.

The heat transfer plate 91 is, for example, a thin plate having an elongated shape (an elongated shape extending in a right and left direction in FIGS. 3 and 4) made of a material such as copper, and a treatment surface 911 as one plate surface faces the holding member 8′ side (the upper side in FIGS. 1 and 2) when the therapeutic energy applying structure 9 is attached to the holding member 8. The treatment surface 911 contacts the body tissue, and the heat transfer plate 91 transfers heat from the flexible substrate 92 to the body tissue (i.e., applies heat energy to the body tissue) when the body tissue is grasped by the holding members 8 and 8′.

The flexible substrate 92 partially generates heat, and functions as a sheet heater that heats the heat transfer plate 91 through such heat generation. As illustrated in FIG. 3 or 4, the flexible substrate 92 includes an insulating substrate 921 and a wiring pattern 922.

The insulating substrate 921 is a sheet having an elongated shape (an elongated shape in the right and left direction in FIGS. 3 and 4) made of polyimide which is an insulating material.

The material of the insulating substrate 921 is not limited to polyimide, and for example, a material having a high heat-resistant insulating property such as aluminum nitride, alumina, glass, and zirconia may be adopted.

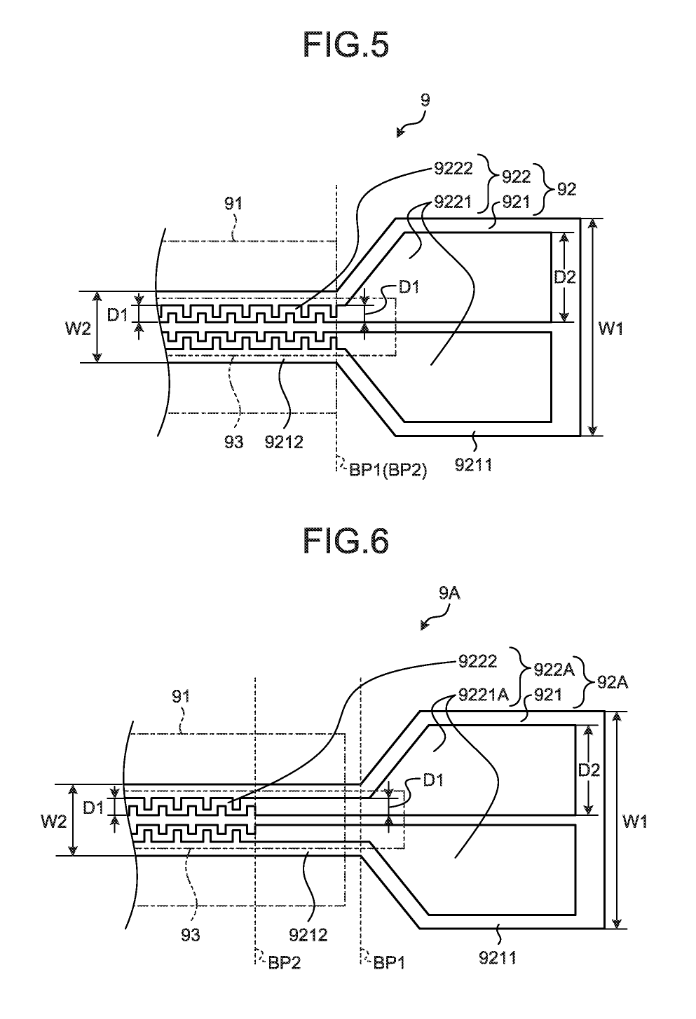

Here, the insulating substrate 921 is formed such that a width W1 (see FIG. 5) on one end side in a longitudinal direction (the right side in FIGS. 3 and 4) is larger than a width W2 (see FIG. 5) on the other end side (the left side in FIGS. 3 and 4). Hereinafter, the one end side will be referred to as a wide region 9211, and the other end side will be referred to as a narrow region 9212.

The wide region 9211 is a region whose width gradually increases from a boundary position BP1 against the narrow region 9212 (see FIG. 5) toward the right side in FIGS. 3 and 4, and further extends to the right side in FIGS. 3 and 4 with the width W1 larger than the width of the heat transfer plate 91.

The narrow region 9212 is a region that extends from the boundary position BP1 against the wide region 9211 to the left side in FIGS. 3 and 4 with the width W2 smaller than the width of the heat transfer plate 91.

The length of the insulating substrate 921 in the right and left direction in FIGS. 3 and 4 is longer than the length of the heat transfer plate 91 in the right and left direction in FIGS. 3 and 4.

The wiring pattern 922 is obtained by processing stainless steel (SUS304), which is a conductive material, and includes a pair of lead wire connection portions 9221 and an electric resistance pattern 9222 (FIG. 4) as illustrated in FIG. 3 or 4. Each thickness of the pair of lead wire connection portions 9221 and the electric resistance pattern 9222 is the same in the first embodiment. The wiring pattern 922 is bonded to one surface of the insulating substrate 921 by thermocompression bonding such that a boundary position BP2 (see FIG. 5) between the pair of lead wire connection portions 9221 and the electric resistance pattern 9222 matches the boundary position BP1 between the wide region 9211 and the narrow region 9212.

The material of the wiring pattern 922 is not limited to stainless steel (SUS304), but may be another stainless material (for example, 400 series), or a conductive material such as platinum or tungsten may be adopted. The wiring pattern 922 may be formed on the one surface of the insulating substrate 921 by evaporation instead of the thermocompression bonding.

The pair of lead wire connection portions 9221 has a function as a connection portion, is provided on the wide region 9211, and faces each other along a width direction of the insulating substrate 921.

The pair of lead wire connection portions 9221 has an outer edge shape following an outer edge shape of the wide region 9211.

Specifically, each of the pair of lead wire connection portions 9221 extends from the boundary position BP2 (boundary position BP1) against the electric resistance pattern 9222 to the right side in FIGS. 3 and 4 with a length D1 (a length along the width direction of the insulating substrate 921 (see FIG. 5)), then, gradually increases in the length toward the right side, and further extends to the right side in FIGS. 3 and 4 with a length D2 (see FIG. 5) larger than the width of the heat transfer plate 91. That is, each of the pair of lead wire connection portions 9221 has a substantially trapezoidal planar shape.

The two lead wires 94 (FIGS. 3 and 4) forming the electric cable C are connected to the pair of lead wire connection portions 9221, respectively.

The electric resistance pattern 9222 has one end that is connected (by conduction) to one of the lead wire connection portions 9221, extends along a U shape following an outer edge shape of the narrow region 9212 while meandering in a wavy shape with a constant line width, and has the other end that is connected (by conduction) to the other of the lead wire connection portions 9221.

Here, a length corresponding to an amplitude of the wavy shape of the electric resistance pattern 9222 is set to the length D1 which is the same as the length D1 in the vicinity of the boundary position BP2 on the pair of lead wire connection portions 9221 (FIG. 5). That is, the pair of lead wire connection portions 9221 has a lower electric resistance value than the electric resistance pattern 9222 since the length D2 thereof is larger than the length D1 of the electric resistance pattern 9222.

The electric resistance pattern 9222 generates heat by applying voltage (by applying current) to the pair of lead wire connection portions 9221 by the control device 3 via the two lead wires 94.

As illustrated in FIGS. 3 and 4, the adhesive sheet (adhesive layer) 93 is provided between the heat transfer plate 91 and the flexible substrate 92, and causes a surface of the heat transfer plate 91 on the opposite side to the treatment surface 911 and one surface (a surface on the wiring pattern 922 side) of the flexible substrate 92 to be adhesively fixed to each other when a part of the flexible substrate 92 protrudes from the heat transfer plate 91. The adhesive sheet 93 is a sheet with an elongated shape (an elongated sheet extending in the right and left direction in FIGS. 3 and 4) that has favorable thermal conductivity and insulating property, is resistant to high temperature, and has an adhesive property, and is formed by, for example, mixing a high thermal conductivity filler (non-conductive material) such as alumina, boron nitride, graphite, and aluminum nitride with resin such as epoxy and polyurethane.

The width of the adhesive sheet 93 is substantially the same as the width W2 of the narrow region 9212. The length (a length in the right and left direction in FIGS. 3 and 4) of the adhesive sheet 93 is longer than the length (the length in the right and left direction in FIGS. 3 and 4) of the heat transfer plate 91 and to be shorter than the length (the length in the right and left direction in FIGS. 3 and 4) of the insulating substrate 921.

Positional Relationship among Heat Transfer Plate, Flexible Substrate, and Adhesive Sheet

Next, a positional relationship among the heat transfer plate 91, the flexible substrate 92, and the adhesive sheet 93 will be described with reference to FIG. 5.

FIG. 5 is a schematic view illustrating the positional relationship between the heat transfer plate 91, the flexible substrate 92, and the adhesive sheet 93. Specifically, FIG. 5 is a schematic view of a proximal end side (the right side in FIGS. 3 and 4) of the therapeutic energy applying structure 9 viewed from the treatment surface 911 side (the upper side in FIGS. 3 and 4 (in a thickness direction of the heat transfer plate 91)).

The heat transfer plate 91 is indicated by a one-dot chain line and the adhesive sheet 93 is indicated by a two-dot chain line in FIG. 5 for convenience of description.

As illustrated in FIG. 5, the heat transfer plate 91 covers the entire region of the electric resistance pattern 9222, and one end of the heat transfer plate 91 in the longitudinal direction (a right end in FIGS. 3 to 5) matches the boundary position BP2 between the pair of lead wire connection portions 9221 and the electric resistance pattern 9222.

As illustrated in FIG. 5, the adhesive sheet 93 covers the entire region of the electric resistance pattern 9222, and one end of the adhesive sheet 93 in the longitudinal direction (a right end in FIGS. 3 to 5) is located on the wide region 9211 side and covers a part of the pair of lead wire connection portions 9221. That is, one end side of the adhesive sheet 93 in the longitudinal direction straddles the boundary position BP2 between the pair of lead wire connection portions 9221 and the electric resistance pattern 9222 and protrudes to the right side in FIG. 5 with respect to the heat transfer plate 91. The two lead wires 94 (FIGS. 3 and 4) are connected, respectively to regions (regions not covered by the adhesive sheet 93) exposed to the outside in the pair of lead wire connection portions 9221.

Configurations of Control Device and Foot Switch

The foot switch 4 is a part that is operated by the operator with a foot. Switching between on and off states is performed to apply current to the medical treatment device 2 (to the electric resistance pattern 9222) from the control device 3 according to the operation using the foot switch 4.

Means for switching between on and off states is not limited to the foot switch 4, and other manually operated switches or the like may be adopted.

The control device 3 includes a central processing unit (CPU) and performs overall control of the medical treatment device 2 according to a predetermined control program. More specifically, the control device 3 applies a voltage to the electric resistance pattern 9222 via the electric cable C (the two lead wires 94) according to the operation of the foot switch 4 performed by the operator (turn-on operation), thereby heating the heat transfer plate 91.

Operation of Medical Treatment Device

Next, an operation of the medical treatment system 1 described above will be described.

The operator grips the medical treatment device 2 and inserts the distal end portion (the grasping portion 7 and a part of the shaft 6) of the medical treatment device 2 into an abdominal cavity through the abdominal wall using, for example, a trocar or the like. The operator operates the operation knob 51 and grasps the body tissue as the treatment target with the holding members 8 and 8′.

Next, the operator operates the foot switch 4 to switch to the on state to apply current from the control device 3 to the medical treatment device 2. When switching to the on state, the control device 3 applies the voltage to the wiring pattern 922 via the electric cable C (the two lead wires 94) to heat the heat transfer plate 91. The body tissue in contact with the heat transfer plate 91 is treated by the heat of the heat transfer plate 91.

In the therapeutic energy applying structure 9 according to the first embodiment described above, the width W1 of the insulating substrate 921 on the one end side (the pair of lead wire connection portions 9221 side) is larger than the width W2 thereof on the other end side (the electric resistance pattern 9222 side). The heat transfer plate 91 covers the entire region of the electric resistance pattern 9222 and is arranged such that the one end in the longitudinal direction matches the boundary position BP2 between the pair of lead wire connection portions 9221 and the electric resistance pattern 9222.

When applying current to the wiring pattern 922 via the two lead wires 94, there is a high possibility that the vicinity of the boundary position BP2 on the pair of lead wire connection portions 9221 is turned into an overheated state. Thus, the heat around the boundary position BP2 on the pair of lead wire connection portions 9221 can be dissipated to the heat transfer plate 91 via the adhesive sheet 93, and further, to the lead wire connection portion 9221 and the insulating substrate 921 (the wide region 9211) by setting the shapes of the insulating substrate 921 and the lead wire connection portion 9221 and the positional relationship between the heat transfer plate 91 and the boundary position BP2 as described above.

Electric resistance values of the pair of lead wire connection portions 9221 are lower than that of the electric resistance pattern 9222. Therefore, it is possible to suppress the heat generation of the pair of lead wire connection portions 9221 itself. As a result, an effect of heat dissipation to the lead wire connection portion 9221 is further enhanced.

As described above, it is possible to avoid the overheated state of the pair of lead wire connection portions 9221 according to the therapeutic energy applying structure 9 of the first embodiment.

The length D2 of the pair of lead wire connection portions 9221 is larger than the length D1 of the electric resistance pattern 9222 in the therapeutic energy applying structure 9 according to the first embodiment. That is, it is possible to set the electric resistance values of the pair of lead wire connection portions 9221 to be low and to suppress the heat generation itself of the pair of lead wire connection portions 9221 by causing the outer edge shapes of the pair of lead wire connection portions 9221 and the electric resistance pattern 9222 to follow the outer edge shape of the insulating substrate 921 and setting the respective lengths D1 and D2.

The adhesive sheet 93 covers the entire region of the electric resistance pattern 9222 and partly protrudes to the pair of lead wire connection portions 9221 side to cover a part of the pair of lead wire connection portions 9221 in the therapeutic energy applying structure 9 according to the first embodiment. That is, the adhesive sheet 93 is arranged so as to straddle the boundary position BP2 between the pair of lead wire connection portions 9221 and the electric resistance pattern 9222. Thus, it is possible to dissipate the heat around the boundary position BP2 on the pair of lead wire connection portions 9221 to the adhesive sheet 93, and to effectively avoid the overheated state of the pair of lead wire connection portions 9221.

### Second Embodiment

Next, a second embodiment of the present invention will be described.

In the following description, the same reference signs are used to designate the same elements as those in the first embodiment, and detailed explanation thereof will be omitted or simplified.

A medical treatment system according to the second embodiment is different from the medical treatment system 1 described in the first embodiment in terms of configurations of the therapeutic energy applying structures 9 and 9′. Each therapeutic energy applying structure provided in each of the holding members 8 and 8′ has the same configuration in the second embodiment. Thus, only the therapeutic energy applying structure provided in the holding member 8 will be described hereinafter.

Configuration of Therapeutic Energy Applying Structure

FIG. 6 is a schematic view illustrating a part of a therapeutic energy applying structure 9A according to the second embodiment of the present invention. Specifically, FIG. 6 is the schematic view corresponding to FIG. 5.

As illustrated in FIG. 6, the therapeutic energy applying structure 9A according to the second embodiment includes a flexible substrate 92A having a wiring pattern 922A with a different shape from that of the wiring pattern 922 of the therapeutic energy applying structure 9 (FIGS. 3 to 5) described in the first embodiment.

Specifically, the wiring pattern 922A includes a pair of lead wire connection portions 9221A which is set such that a part with the length D1 is longer than that of the pair of lead wire connection portions 9221 in the right and left direction in FIG. 6. Thus, the boundary position BP2 between the pair of lead wire connection portions 9221A and the electric resistance pattern 9222 is located on the narrow region 9212 in the therapeutic energy applying structure 9A as illustrated in FIG. 6.

The length D2 is larger than the length D1 of the electric resistance pattern 9222 even in the above-described case of adopting the pair of lead wire connection portions 9221A, and thus, the pair of lead wire connection portions 9221A has a lower electric resistance value than the electric resistance pattern 9222, which is similar to the first embodiment.

In the therapeutic energy applying structure 9A illustrated in FIG. 6, the heat transfer plate 91 covers the entire region of the electric resistance pattern 9222, and one end of the heat transfer plate 91 in the longitudinal direction (a right end in FIG. 6) is located on the pair of lead wire connection portions 9221A side within the narrow region 9212. That is, the heat transfer plate 91 is arranged so as to straddle the boundary position BP2 between the pair of lead wire connection portions 9221A and the electric resistance pattern 9222.

Even if the pair of lead wire connection portions 9221A is adopted and the heat transfer plate 91 is arranged so as to straddle the boundary position BP2 between the pair of lead wire connection portions 9221A and the electric resistance pattern 9222 as in the second embodiment, it is possible to dissipate the heat around the boundary position BP2 on the pair of lead wire connection portions 9221A to the heat transfer plate 91 via the adhesive sheet 93 and further dissipate the heat to the lead wire connection portions 9221A and the insulating substrate 921 (wide region 9211). Hence, the same advantageous effects as those of the first embodiment can be obtained.

### Third Embodiment

Next, a third embodiment of the present invention will be described.

In the following description, the same reference signs are used to designate the same elements as those in the first embodiment, and detailed explanation thereof will be omitted or simplified.

A medical treatment system according to the third embodiment is different from the medical treatment system 1 described in the first embodiment in terms of configurations of the therapeutic energy applying structures 9 and 9′. Each therapeutic energy applying structure provided in each of the holding members 8 and 8′ has the same configuration in the third embodiment. Thus, only the therapeutic energy applying structure provided in the holding member 8 will be described hereinafter.

Configuration of Therapeutic Energy Applying Structure

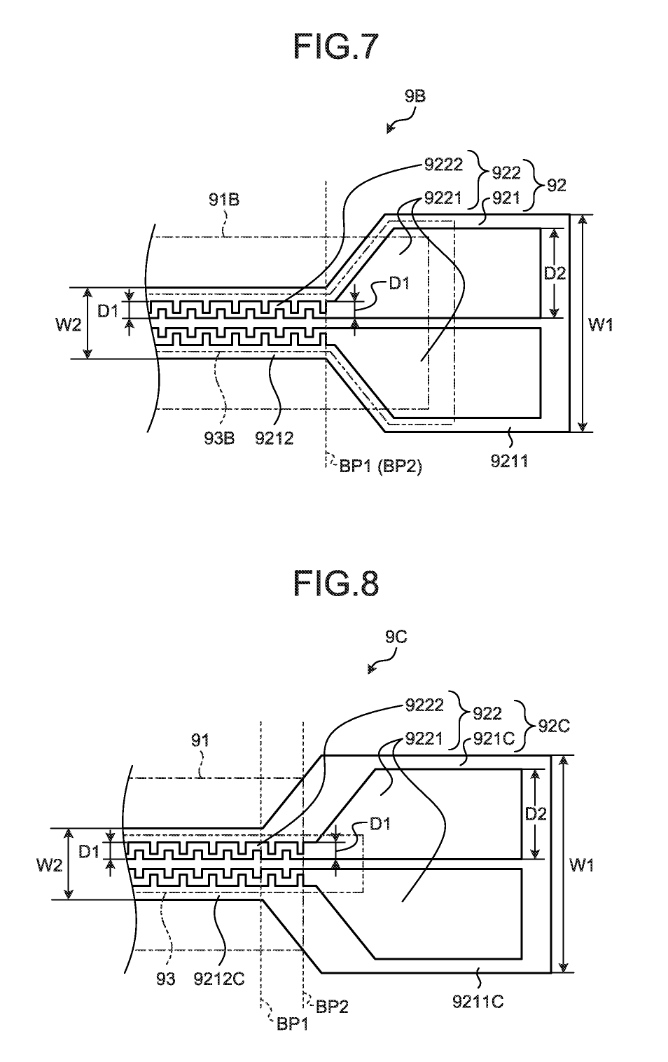

FIG. 7 is a schematic view illustrating a part of a therapeutic energy applying structure 9B according to the third embodiment of the present invention. Specifically, FIG. 7 is a schematic view corresponding to FIG. 5.

As illustrated in FIG. 7, the therapeutic energy applying structure 9B according to the third embodiment includes a heat transfer plate 91B having a different length in the right and left direction in FIG. 7 from that of the heat transfer plate 91 and includes an adhesive sheet 93B having a different shape from that of the adhesive sheet 93, compared to the therapeutic energy applying structure 9 (FIGS. 3 to 5) described in the first embodiment.

Specifically, the heat transfer plate 91B is set to have the length (the length in the right and left direction in FIG. 7) that is longer than that of the heat transfer plate 91. The heat transfer plate 91B covers the entire region of the electric resistance pattern 9222, and one end of the heat transfer plate 91B in the longitudinal direction (a right end in FIG. 7) is located on the wide region 9211 side and covers a part of the pair of lead wire connection portions 9221 as illustrated in FIG. 7. That is, the heat transfer plate 91B is arranged so as to straddle the boundary position BP2 between the pair of lead wire connection portions 9221 and the electric resistance pattern 9222.

In order to avoid electrical contact between the heat transfer plate 91B and the wiring pattern 922, the adhesive sheet 93B is formed such that one end side (the right side in FIG. 7) in the longitudinal direction has a wide width to cover the boundary position BP2 side on the pair of lead wire connection portions 9221 as illustrated in FIG. 7.

Even if the heat transfer plate 91B and the adhesive sheet 93B are adopted and the heat transfer plate 91B and the adhesive sheet 93B are disposed so as to straddle the boundary position BP2 between the pair of lead wire connection portions 9221 and the electric resistance pattern 9222 as in the third embodiment, it is possible to dissipate the heat around the boundary position BP2 on the pair of lead wire connection portions 9221 to the heat transfer plate 91B via the adhesive sheet 93B and further dissipate the heat to the lead wire connection portions 9221 and the insulating substrate 921 (wide region 9211). Hence, the same advantageous effects as those of the first embodiment can be obtained.

### Fourth Embodiment

Next, a fourth embodiment of the present invention will be described.

In the following description, the same reference signs are used to designate the same elements as those in the first embodiment, and detailed explanation thereof will be omitted or simplified.

A medical treatment system according to the fourth embodiment is different from the medical treatment system 1 described in the first embodiment in terms of configurations of the therapeutic energy applying structures 9 and 9′. Each therapeutic energy applying structure provided in each of the holding members 8 and 8′ has the same configuration in the fourth embodiment. Thus, only the therapeutic energy applying structure provided in the holding member 8 will be described hereinafter.

Configuration of Therapeutic Energy Applying Structure

FIG. 8 is a schematic view illustrating a part of a therapeutic energy applying structure 9C according to the fourth embodiment of the present invention. Specifically, FIG. 8 is a schematic view corresponding to FIG. 5.

As illustrated in FIG. 8, the therapeutic energy applying structure 9C according to the fourth embodiment includes a flexible substrate 92C having an insulating substrate 921C with a different shape from that of the insulating substrate 921 of the therapeutic energy applying structure 9 (FIGS. 3 to 5) described in the first embodiment.

Specifically, the insulating substrate 921C has a wide region 9211C with a longer length in the right and left direction in FIG. 8 than the wide region 9211 and a narrow region 9212C with a shorter length in the right and left direction in FIG. 8 than the narrow region 9212 while having the entire length (the length in the right and left direction in FIG. 8) that is the same as that of the insulating substrate 921. That is, the boundary position BP2 between the pair of lead wire connection portions 9221 and the electric resistance pattern 9222 is located on the right side of the boundary position BP1 between the wide region 9211C and the narrow region 9212C, and located within the wide region 9211C as illustrated in FIG. 8.

Even if the insulating substrate 921C is adopted and the boundary position BP2 between the pair of lead wire connection portions 9221 and the electric resistance pattern 9222 is located within the wide region 9211C as in the fourth embodiment, it is possible to dissipate the heat around the boundary position BP2 on the pair of lead wire connection portions 9221 to the heat transfer plate 91 via the adhesive sheet 93 and further dissipate the heat to the lead wire connection portions 9221 and the insulating substrate 921 (wide region 9211C). Hence, the same advantageous effects as those of the first embodiment can be obtained

### Fifth Embodiment

Next, a fifth embodiment of the present invention will be described.

In the following description, the same reference signs are used to designate the same elements as those in the first embodiment, and detailed explanation thereof will be omitted or simplified.

A medical treatment system according to the fifth embodiment is different from the medical treatment system 1 described in the first embodiment in terms of configurations of the therapeutic energy applying structures 9 and 9′. Each therapeutic energy applying structure provided in each of the holding members 8 and 8′ has the same configuration in the fifth embodiment. Thus, only the therapeutic energy applying structure provided in the holding member 8 will be described hereinafter.

Configuration of Therapeutic Energy Applying Structure

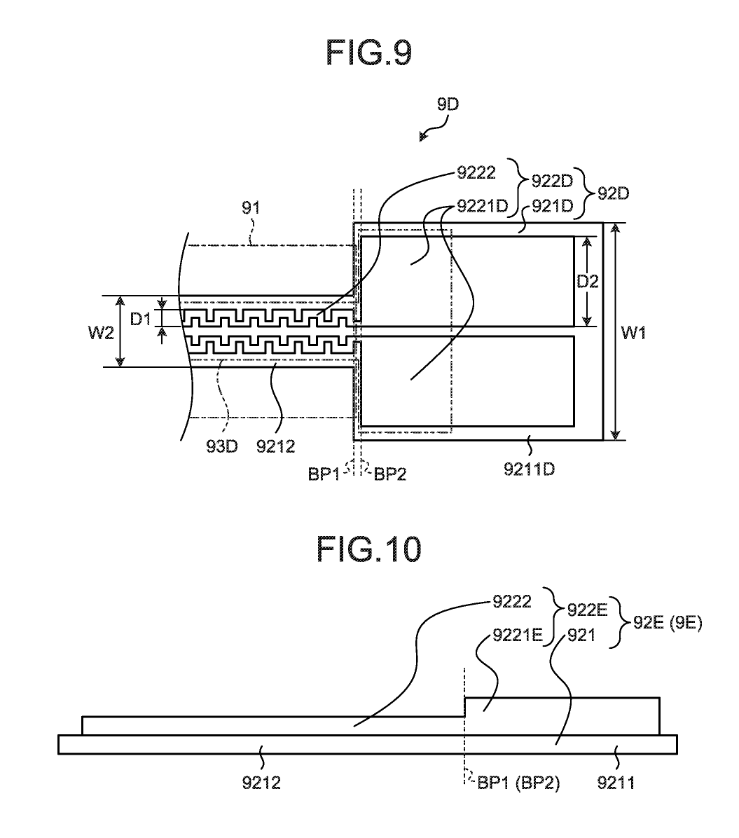

FIG. 9 is a schematic view illustrating a part of a therapeutic energy applying structure 9D according to the fifth embodiment of the present invention. Specifically, FIG. 9 is a schematic view corresponding to FIG. 5.

As illustrated in FIG. 9, the therapeutic energy applying structure 9D according to the fifth embodiment includes a flexible substrate 92D having an insulating substrate 921D and a wiring pattern 922D with different shapes from those of the insulating substrate 921 and the wiring pattern 922, and includes an adhesive sheet 93D having a different shape from that of the adhesive sheet 93, compared to the therapeutic energy applying structure 9 (FIGS. 3 to 5) described in the first embodiment.

Specifically, the insulating substrate 921D has the rectangular wide region 9211D, which has the width W1 at the boundary position BP1 and extends to the right side in FIG. 9 from the boundary position BP1 with the width W1 as illustrated in FIG. 9, which is different from the wide region 9211.

As illustrated in FIG. 9, the wiring pattern 922D has the pair of rectangular lead wire connection portions 9221D each of which has the length D2 at the boundary position BP2 and extends to the right side in FIG. 9 from the boundary position BP2 with the length D2, which is different from the pair of lead wire connection portions 9221.

The length D2 is larger than the length D1 of the electric resistance pattern 9222 even in the above-described case of adopting the pair of lead wire connection portions 9221D, and thus, the pair of lead wire connection portions 9221D has a lower electric resistance value than the electric resistance pattern 9222, which is similar to the first embodiment.

Here, the wiring pattern 922D is formed such that the boundary position BP2 is located within the wide region 9211D in the fifth embodiment. The heat transfer plate 91 covers the entire region of the electric resistance pattern 9222, and one end of the heat transfer plate 91 in the longitudinal direction (a right end in FIG. 9) matches the boundary position BP2. In FIG. 9, the one end of the heat transfer plate 91 in the longitudinal direction is shifted from the boundary position BP2 for the purpose of illustration.

In order to avoid electrical contact between the heat transfer plate 91 and the wiring pattern 922D, the adhesive sheet 93D is formed such that one end side (the right side in FIG. 9) in the longitudinal direction has a wide width to cover the boundary position BP2 side on the pair of lead wire connection portions 9221D as illustrated in FIG. 9.

Even if the flexible substrate 92D (the insulating substrate 921D and the wiring pattern 922D) and the adhesive sheet 93D are adopted as in the fifth embodiment, it is possible to dissipate the heat around the boundary position BP2 on the pair of lead wire connection portions 9221D to the heat transfer plate 91 via the adhesive sheet 93D and further dissipate the heat to the lead wire connection portions 9221D and the insulating substrate 921D (wide region 9211D). Hence, the same advantageous effects as those of the first embodiment can be obtained.

In particular, the heat capacities of the wide region 9211D and the pair of lead wire connection portions 9221D are increased, and thus, it is possible to further enhance the heat dissipation effect of the pair of lead wire connection portions 9221D.

### Sixth Embodiment

Next, a sixth embodiment of the present invention will be described.

In the following description, the same reference signs are used to designate the same elements as those in the first embodiment, and detailed explanation thereof will be omitted or simplified.

A medical treatment system according to the sixth embodiment is different from the medical treatment system 1 described in the first embodiment in terms of configurations of the therapeutic energy applying structures 9 and 9′. Each therapeutic energy applying structure provided in each of the holding members 8 and 8′ has the same configuration in the sixth embodiment. Thus, only the therapeutic energy applying structure provided in the holding member 8 will be described hereinafter.

Configuration of Therapeutic Energy Applying Structure

FIG. 10 is a side view illustrating a flexible substrate 92E forming a therapeutic energy applying structure 9E according to the sixth embodiment of the present invention.

As illustrated in FIG. 10, the therapeutic energy applying structure 9E according to the sixth embodiment includes the flexible substrate 92E having a wiring pattern 922E with a different shape from that of the wiring pattern 922 of the therapeutic energy applying structure 9 (FIGS. 3 to 5) described in the first embodiment.

Specifically, the wiring pattern 922E includes a pair of lead wire connection portions 9221E having a larger thickness than the pair of lead wire connection portions 9221. In other words, the wiring pattern 922E is formed such that the thickness of the pair of lead wire connection portions 9221E is larger than that of the electric resistance pattern 9222. That is, the length D2 of the pair of lead wire connection portions 9221E is larger than the length D1 of the electric resistance pattern 9222 and the thickness thereof is larger than the thickness of the electric resistance pattern 9222, and thus, the pair of lead wire connection portions 9221E has an electric resistance value that is even lower than that of the electric resistance pattern 9222.

This wiring pattern 922E can be manufactured by, for example, uniformly forming the total thickness of the wiring pattern to be relatively large, and then, performing etching on a portion of the electric resistance pattern to reduce the thickness of the portion.

Although the total thickness of the pair of lead wire connection portions 9221E is larger than the thickness of the electric resistance pattern 9222 in FIG. 10, the present invention is not limited thereto, and only a part of the pair of lead wire connection portions 9221E may be configured to be larger than the thickness of the electric resistance pattern 9222.

Although the heat transfer plate 91 and the adhesive sheet 93 are not illustrated in FIG. 10, the positional relationship between the heat transfer plate 91 and the adhesive sheet 93 with respect to the boundary position BP1 between the wide region 9211 and the narrow region 9212 and the boundary position BP2 between the pair of lead wire connection portions 9221E and the electric resistance pattern 9222 is the same as that in the first embodiment.

According to the sixth embodiment, not only the same advantageous effects as those in the first embodiment but also the following advantageous effects can be obtained.

In the therapeutic energy applying structure 9E according to the sixth embodiment, the thickness of the pair of lead wire connection portions 9221E is larger than the thickness of the electric resistance pattern 9222. Thus, it is possible to further lower the electric resistance value of the pair of lead wire connection portions 9221E, and to further suppress heat generation itself of the pair of lead wire connection portions 9221E. Further, a heat radiation effect of the lead wire connection portion 9221E can be enhanced based on such a result.

### Seventh Embodiment

Next, a seventh embodiment of the present invention will be described.

In the following description, the same reference signs are used to designate the same elements as those in the first embodiment, and detailed explanation thereof will be omitted or simplified.

A medical treatment system according to the seventh embodiment is different from the medical treatment system 1 described in the first embodiment in terms of configurations of the therapeutic energy applying structures 9 and 9′. Each therapeutic energy applying structure provided in each of the holding members 8 and 8′ has the same configuration in the seventh embodiment. Thus, only the therapeutic energy applying structure provided in the holding member 8 will be described hereinafter.

Configuration of Therapeutic Energy Applying Structure

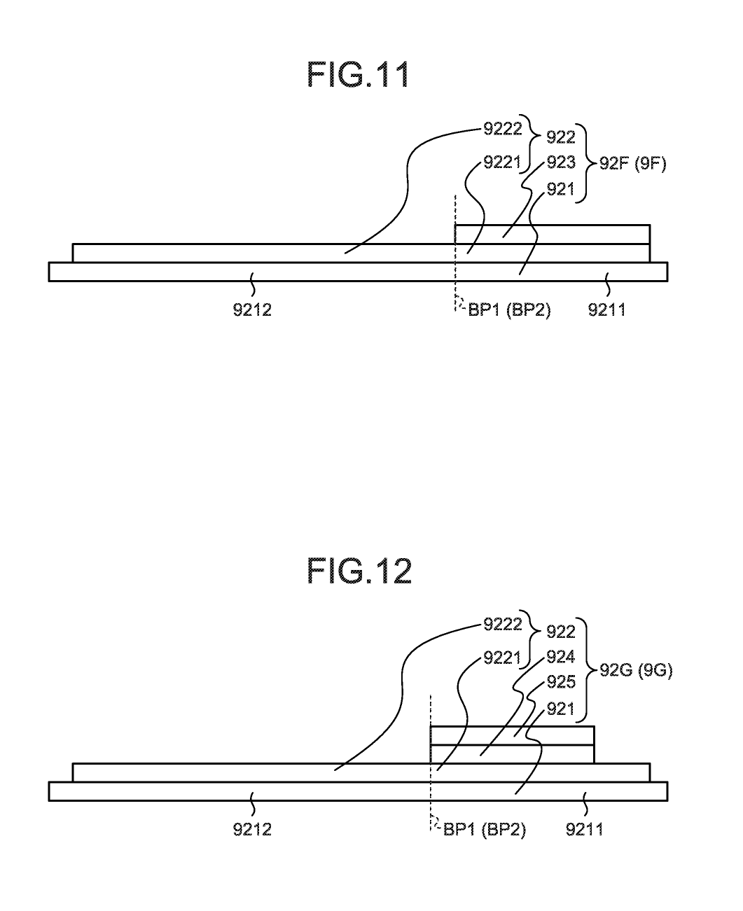

FIG. 11 is a side view illustrating a flexible substrate 92F forming a therapeutic energy applying structure 9F according to the seventh embodiment of the present invention.

As illustrated in FIG. 11, the therapeutic energy applying structure 9F according to the seventh embodiment includes the flexible substrate 92F additionally having a pair of conductive layers 923, compared to the therapeutic energy applying structure 9 (FIGS. 3 to 5) described in the first embodiment.

Specifically, the pair of conductive layers 923 is a layer that is made of a conductive material such as gold, silver, copper, and nickel and formed by plating or electroforming on the entire surface on each of the pair of lead wire connection portions 9221. The two lead wires 94 (FIGS. 3 and 4) are connected to the pair of conductive layers 923, respectively.

In the seventh embodiment, the pair of lead wire connection portions 9221 corresponds to a connection portion main body, and the pair of conductive layers 923 corresponds to a conductive portion. The pair of lead wire connection portions 9221 and the pair of conductive layers 923 constitute a connection portion. That is, the pair of lead wire connection portions 9221 and the pair of conductive layers 923 corresponding to the connection portion have the length D2 larger than the length D1 of the electric resistance pattern 9222 and have the larger thickness than the thickness of the electric resistance pattern 9222 by a thickness of the pair of conductive layers 923, and thus, have an electric resistance value that is even lower than that of the electric resistance pattern 9222.

Although the pair of conductive layers 923 is formed on the entire surface of the pair of lead wire connection portions 9221 in FIG. 11, the present invention is not limited thereto, and the pair of conductive layers 923 may be configured to be formed on only a part of each of the pair of lead wire connection portions 9221.

The heat transfer plate 91 and the adhesive sheet 93 are not illustrated in FIG. 11, but a positional relationship between the heat transfer plate 91 and the adhesive sheet 93 with respect to the boundary position BP1 between the wide region 9211 and the narrow region 9212 and the boundary position BP2 between the pair of lead wire connection portions 9221 and the electric resistance pattern 9222 is the same as that in the first embodiment.

Since the pair of conductive layers 923 is provided on the pair of lead wire connection portions 9221, respectively, and the total thickness of the pair of lead wire connection portions 9221 and the pair of conductive layers 923 is made larger than the thickness of the electric resistance pattern 9222 in the therapeutic energy applying structure 9F according to the seventh embodiment described above, the same advantageous effects as those of the sixth embodiment can be obtained.

### Eighth Embodiment

Next, an eighth embodiment of the present invention will be described.

In the following description, the same reference signs are used to designate the same elements as those in the first embodiment, and detailed explanation thereof will be omitted or simplified.

A medical treatment system according to the eighth embodiment is different from the medical treatment system 1 described in the first embodiment in terms of configurations of the therapeutic energy applying structures 9 and 9′. Each therapeutic energy applying structure provided in each of the holding members 8 and 8′ has the same configuration in the eighth embodiment. Thus, only the therapeutic energy applying structure provided in the holding member 8 will be described hereinafter.

Configuration of Therapeutic Energy Applying Structure

FIG. 12 is a side view illustrating a flexible substrate 92G forming a therapeutic energy applying structure 9G according to the eighth embodiment of the present invention.

As illustrated in FIG. 12, the therapeutic energy applying structure 9G according to the eighth embodiment includes the flexible substrate 92G additionally having a pair of first conductive portions 924 and a pair of insulating portions 925, compared to the therapeutic energy applying structure 9 (FIGS. 3 to 5) described in the first embodiment.

Specifically, the pair of insulating portions 925 is a plate body made of an insulating material such as polyimide. The pair of first conductive portions 924 is a plate body made of a conductive material such as copper, and is attached to one plate surface of each of the pair of insulating portions 925.

The pair of first conductive portions 924 and the pair of insulating portions 925 have substantially the same planar shape as the planar shape of the pair of lead wire connection portions 9221, but a proximal end side (the right side in FIG. 12) thereof is shorter than the pair of lead wire connection portions 9221 in order to secure a region to bond the two lead wires 94 to the pair of lead wire connection portions 9221.

The pair of insulating portions 925 to which the pair of first conductive portions 924 is affixed, respectively, is bonded by diffusion bonding, ultrasonic welding, or resistance welding when the pair of first conductive portions 924 faces the pair of lead wire connection portions 9221. The pair of conductive layers 923 described in the seventh embodiment may be provided between the pair of first conductive portions 924 and the pair of lead wire connection portions 9221, respectively, as necessary at the time of bonding. Alternatively, solder, a conductive adhesive or the like may be employed to firmly bond the respective portions.

In the eighth embodiment, the pair of lead wire connection portions 9221 corresponds to the connection portion main body, and the pair of first conductive portions 924 corresponds to the conductive portion. The pair of lead wire connection portions 9221 and the pair of first conductive portions 924 constitute the connection portion. That is, the pair of lead wire connection portions 9221 and the pair of first conductive portions 924 corresponding to the connection portion have the length D2 larger than the length D1 of the electric resistance pattern 9222 and have the larger thickness than the thickness of the electric resistance pattern 9222 by a thickness of the pair of first conductive portions 924, and thus, have an electric resistance value that is even lower than that of the electric resistance pattern 9222.

The heat transfer plate 91 and the adhesive sheet 93 are not illustrated in FIG. 12, but a positional relationship between the heat transfer plate 91 and the adhesive sheet 93 with respect to the boundary position BP1 between the wide region 9211 and the narrow region 9212 and the boundary position BP2 between the pair of lead wire connection portions 9221 and the electric resistance pattern 9222 is the same as that in the first embodiment.

Since the pair of first conductive portions 924 is provided on the pair of lead wire connection portions 9221, respectively, and the total thickness of the pair of lead wire connection portions 9221 and the pair of first conductive portions 924 is made larger than the thickness of the electric resistance pattern 9222 in the therapeutic energy applying structure 9G according to the eighth embodiment described above, the same advantageous effects as those of the sixth embodiment can be obtained.

In addition, since the pair of insulating portions 925 are affixed onto the pair of first conductive portions 924, respectively, it is possible to omit insulating treatment of the pair of lead wire connection portions 9221 at the time of manufacturing the therapeutic energy applying structure 9G.

### Ninth Embodiment

### Next, a ninth embodiment of the present invention will be described.

In the following description, the same reference signs are used to designate the same elements as those in the first embodiment, and detailed explanation thereof will be omitted or simplified.

A medical treatment system according to the ninth embodiment is different from the medical treatment system 1 described in the first embodiment in terms of configurations of the therapeutic energy applying structures 9 and 9′. Each therapeutic energy applying structure provided in each of the holding members 8 and 8′ has the same configuration in the ninth embodiment. Thus, only the therapeutic energy applying structure provided in the holding member 8 will be described hereinafter.

Configuration of Therapeutic Energy Applying Structure

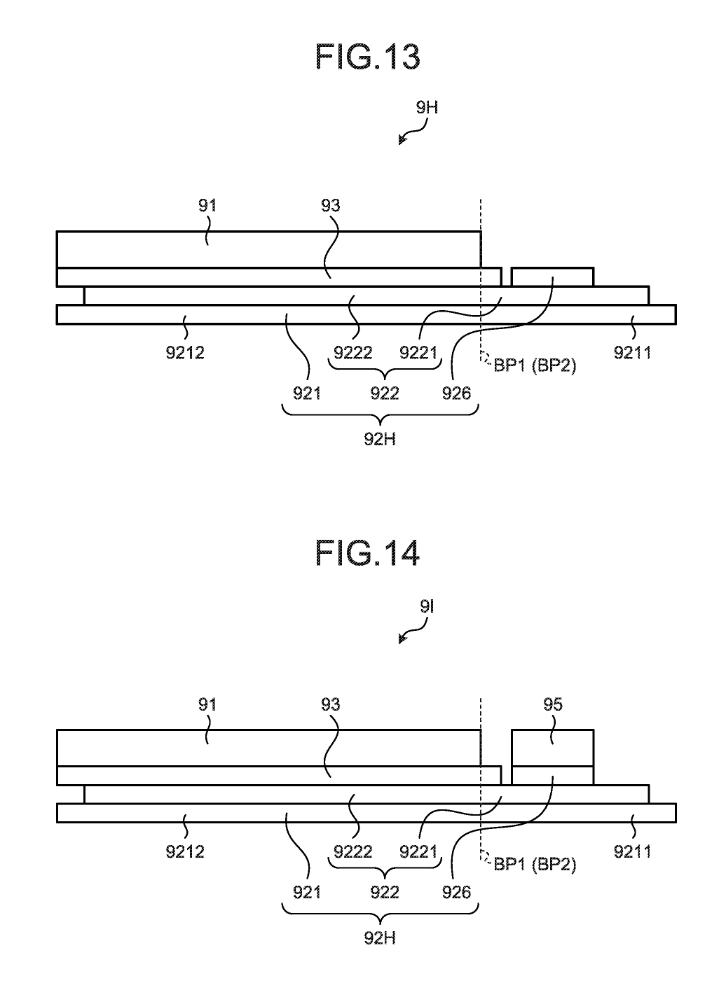

FIG. 13 is a side view illustrating a therapeutic energy applying structure 9H according to the ninth embodiment of the present invention.

As illustrated in FIG. 13, the therapeutic energy applying structure 9H according to the ninth embodiment includes a flexible substrate 92H additionally having a second conductive portion 926, compared to the therapeutic energy applying structure 9 (FIGS. 3 to 5) described in the first embodiment.

Specifically, the second conductive portion 926 is an adhesive sheet similar to the adhesive sheet 93, and is affixed onto the pair of lead wire connection portions 9221 straddling the pair of lead wire connection portions 9221. The two lead wires 94 (FIGS. 3 and 4) are bonded to regions of the pair of lead wire connection portions 9221 exposed to the outside (the regions on a proximal end side (the right side in FIG. 13) that are not covered by the second conductive portion 926).

In the ninth embodiment, the pair of lead wire connection portions 9221 corresponds to the connection portion main body, and the second conductive portion 926 corresponds to the conductive portion. The pair of lead wire connection portions 9221 and the second conductive portion 926 correspond to the connection portion. That is, the pair of lead wire connection portions 9221 and the second conductive portion 926 corresponding to the connection portion have the length D2 larger than the length D1 of the electric resistance pattern 9222 and have the larger thickness than the thickness of the electric resistance pattern 9222 by a thickness of the second conductive portion 926, and thus, have an electric resistance value that is even lower than that of the electric resistance pattern 9222.

As illustrated in FIG. 13, the positional relationship between the heat transfer plate 91 and the adhesive sheet 93 with respect to the boundary position BP1 between the wide region 9211 and the narrow region 9212 and the boundary position BP2 between the pair of lead wire connection portions 9221 and the electric resistance pattern 9222 is the same as that in the first embodiment. The adhesive sheet 93 is arranged such that one end in the longitudinal direction (a right end in FIG. 13) is spaced apart from the second conductive portion 926 as illustrated in FIG. 13.

Since the second conductive portion 926 is provided on the pair of lead wire connection portions 9221, and the total thickness of the pair of lead wire connection portions 9221 and the second conductive portion 926 is made larger than the thickness of the electric resistance pattern 9222 in the therapeutic energy applying structure 9H according to the ninth embodiment described above, the same advantageous effects as those of the sixth embodiment can be obtained.

In addition, since the second conductive portion 926 is arranged so as to be separated from the adhesive sheet 93, heat transferred from the wiring pattern 922 to the adhesive sheet 93 is not transferred to the second conductive portion 926. That is, it is possible to effectively dissipate the heat of the pair of lead wire connection portions 9221 using the second conductive portion 926.

### Modified Example of Ninth Embodiment

FIG. 14 is a schematic view illustrating a modified example of the ninth embodiment of the present invention. Specifically, FIG. 14 is a schematic view corresponding to FIG. 13.

A therapeutic energy applying structure 91 illustrated in FIG. 14 may be adopted instead of the therapeutic energy applying structure 9H described in the ninth embodiment.

Specifically, the therapeutic energy applying structure 91 has a structure in which a heat sink 95 made of metal such as aluminum, copper, and iron or ceramic having high heat conductivity such as aluminum nitride is bonded onto a top surface of the second conductive portion 926 as illustrated in FIG. 14.

Coating having a heat dissipation effect may be applied on the top surface of the second conductive portion 926 instead of forming the heat sink 95. For example, it is possible to exemplify diamond-like carbon (DLC), alumina, and the like, or a coating material having a high emissivity, an alumite process, and the like as the coating. The second conductive portion 926 may be omitted, and the above-described coating may be applied on the pair of lead wire connection portions 9221.

### Other Embodiments

The present invention is not limited only to the first to ninth embodiments and the modified example of the ninth embodiment.

In the first to ninth embodiments and the modified example of the ninth embodiment, the therapeutic energy applying structures 9 (9′) and 9A to 9I are provided on both of the holding members 8 and 8′, respectively. Alternatively, the therapeutic energy applying structure may be provided only on one of the holding members 8 and 8′.

In the first to ninth embodiments and the modified example of the ninth embodiment, the therapeutic energy applying structures 9 (9′) and 9A to 9I are configured to apply heat energy to the body tissue. Besides the heat energy, applying high-frequency energy or ultrasound energy to the body tissue may be adopted.

According to the therapeutic energy applying structure and the medical treatment device of some embodiments, it is possible to avoid an overheated state of a connection portion.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A therapeutic energy applying structure comprising:
an insulating substrate;
an electric resistance pattern provided on one surface of the insulating substrate and configured to generate heat by applying current;
a connection portion provided on the one surface of the insulating substrate and configured to be electrically connected to the electric resistance pattern, the connection portion having a lower electric resistance value than the electric resistance pattern; and
a heat transfer plate disposed so as to face the one surface of the insulating substrate and configured to transfer the heat from the electric resistance pattern to a body tissue, wherein
each of the insulating substrate and the heat transfer plate has an elongated shape extending in a same direction,
the electric resistance pattern and the connection portion are arranged side by side in a longitudinal direction of the insulating substrate,
the insulating substrate has a first region on which the connection portion is provided and has a second region on which the electric resistance pattern is provided,
a width of the first region is larger than a width of the second region,
the heat transfer plate covers an entire region of the electric resistance pattern when viewed from a thickness direction of the heat transfer plate, and
one end of the heat transfer plate in the longitudinal direction matches a boundary position between the electric resistance pattern and the connection portion, or the one end of the heat transfer plate is located on the connection portion deviating from the boundary position.

2. The therapeutic energy applying structure according to claim 1, wherein
a length of the connection portion in a transverse direction of the insulating substrate is larger than a length of the electric resistance pattern in the transverse direction.

3. The therapeutic energy applying structure according to claim 2, wherein
the electric resistance pattern and the connection portion have outer edges following an outer edge of the insulating substrate.

4. The therapeutic energy applying structure according to claim 1, wherein
a boundary between the first and second regions of the insulating substrate corresponds to the boundary position, and
the one end of the heat transfer plate in the longitudinal direction matches the boundary position when viewed from the thickness direction of the heat transfer plate.

5. The therapeutic energy applying structure according to claim 1, further comprising an adhesive layer provided between the one surface of the insulating substrate and the heat transfer plate to adhesively fix the insulating substrate and the heat transfer plate to each other, wherein
the adhesive layer covers the entire region of the electric resistance pattern and partly protrudes to the connection portion to cover a part of the connection portion.

6. The therapeutic energy applying structure according to claim 1, wherein
a thickness of at least a part of the connection portion is larger than a thickness of the electric resistance pattern.

7. The therapeutic energy applying structure according to claim 6, wherein
the connection portion comprises:
a connection portion main body having the same thickness as the electric resistance pattern; and
a conductive portion containing a conductive material and provided on at least a part of the connection portion main body.

8. A medical treatment device comprising the therapeutic energy applying structure according to claim 1.

9. A therapeutic energy applying structure comprising:
an insulating substrate;
an electric resistance pattern provided on one surface of the insulating substrate and configured to generate heat by applying current;
a connection portion provided on the one surface of the insulating substrate and configured to be electrically connected to the electric resistance pattern, the connection portion having a lower electric resistance value than the electric resistance pattern; and
a heat transfer plate disposed so as to face the one surface of the insulating substrate and configured to transfer the heat from the electric resistance pattern to a body tissue, wherein
each of the insulating substrate and the heat transfer plate has an elongated shape extending in a same direction,
the electric resistance pattern and the connection portion are arranged side by side in a longitudinal direction of the insulating substrate,
the insulating substrate has a first region on which the connection portion is provided and has a second region on which the electric resistance pattern is provided, and
a width of the first region is larger than a width of the second region.
